SEQUENCE LISTING (15 sequences)

SEQ ID NO: 1 - protein, 4 aa, Artificial Sequence
  Syntentic peptide
KRSR

SEQ ID NO: 2 - protein, 4 aa, Artificial Sequence
  Synthetic peptide
KSRR

SEQ ID NO: 3 - protein, 9 aa, Artificial Sequence
  Synthetic peptide
KRSRGGGGY

SEQ ID NO: 4 - protein, 9 aa, Artificial Sequence
  Synthetic peptide
KSRRGGGGY

SEQ ID NO: 5 - protein, 10 aa, Artificial Sequence
  Synthetic peptide
KPSSAPTQLN

SEQ ID NO: 6 - protein, 10 aa, Artificial Sequence
  Synthetic peptide
AISVLYFDDS

SEQ ID NO: 7 - protein, 10 aa, Artificial Sequence
  Synthetic peptide
SNVILKKYRN

SEQ ID NO: 8 - protein, 14 aa, Artificial Sequence
  Synthetic peptide
KRSRSNVILKKYRN

SEQ ID NO: 9 - protein, 18 aa, Artificial Sequence
  Synthetic peptide
KRSRGGGGKPSSAPTQLN

SEQ ID NO: 10 - protein, 18 aa, Artificial Sequence
  Synthetic peptide
KRSRGGGGAISVLYFDDS

SEQ ID NO: 11 - protein, 18 aa, Artificial Sequence
  Synthetic peptide
KRSRGGGGSNVILKKYRN

SEQ ID NO: 12 - protein, 28 aa, Artificial Sequence
  Synthetic peptide
KRSRGGGGKPSSAPTQLNAISVLYFDDS

SEQ ID NO: 13 - protein, 28 aa, Artificial Sequence
  Synthetic peptide
KRSRGGGGAISVLYFDDSSNVILKKYRN

SEQ ID NO: 14 - protein, 38 aa, Artificial Sequence
  Synthetic peptide
KRSRGGGGKPSSAPTQLNAISVLYFDDSSNVILKKYRN

SEQ ID NO: 15 - protein, 4 aa, Artificial Sequence
  Synthetic peptide
RGDS

(19) United States
(12) Patent Application Publication
(10) Pub. No.: US 2013/0337028 A1
(43) Pub. Date: Dec. 19, 2013

(54) BIOMIMETIC PEPTIDES FOR BONE AUGMENTATION

(71) Applicants: Ganesan Balasundaram, Osseo, MN (US); Matthew Hedrick, Carmel, IN (US)

(72) Inventors: Ganesan Balasundaram, Osseo, MN (US); Matthew Hedrick, Carmel, IN (US)

(73) Assignee: NANOVIS, INC., West Lafayette, IN (US)

(21) Appl. No.: 13/704,795

(22) PCT Filed: Jun. 22, 2011
(86) PCT No.: PCT/US11/41497

§ 371 Date: Jul. 25, 2013

Related U.S. Application Data
(60) Provisional application No. 61/358,859, filed on Jun. 25, 2010.

(51) Int. Cl. C07K 14/00; A61L 27/54; C07K 7/08; A61L 27/22; C07K 5/10; C07K 7/06

(52) U.S. Cl. CPC C07K 14/001; C07K 5/10; C07K 7/06; C07K 7/08; A61L 27/22; A61L 27/54
USPC 424/422; 530/330; 530/328; 530/327; 530/326; 530/324; 530/322; 514/16.7; 435/375

(57) ABSTRACT

The present invention relates to synthetic peptides for use in bone tissue repair and regeneration applications. The present invention also relates to various compositions and devices (including implantable orthopedic/dental devices) that contain the synthetic peptides of the present invention, and methods involving the use of the synthetic peptides of the present invention. The present invention also relates to a bone replacement or bone-reconstructive material, which includes a polymer matrix and a synthetic peptide of the present invention.

### CROSS-REFERENCE TO RELATED APPLICATIONS

The present application claims the benefit of U.S. Provisional Patent Application Ser. No. 61/358,859, filed Jun. 25, 2010, the entire disclosure of which is incorporated by reference herein.

### FIELD OF THE INVENTION

The present invention relates to synthetic peptides for use in bone tissue repair and regeneration applications. The present invention also relates to various compositions and devices that contain the synthetic peptides of the present invention, and methods involving the use of the synthetic peptides of the present invention.

### BACKGROUND OF THE INVENTION

Conventional treatment methods for bone tissue repair and regeneration include the use of implants for orthopedic and dental applications. Many of these conventional methods involve implants made of metals and metal alloys, which are typically selected based on mechanical properties (e.g., primarily strength under loading. Unfortunately, the use of conventional metals and metal alloys that meet mechanical requirements for bone replacements can result in metal material failure under long-term physiological loading, necessitating the surgical removal of failed bone implants. Traditional ceramics have long been appreciated for their cytocompatibility.

Conventional ceramic formulations of materials such as hydroxyapatite, bioglasses, bioactive glass ceramics, and calcium phosphate have been shown to enhance formation of new bone mineralized matrix. “Conventional” refers to ceramics having a grain size greater than 100 nm. In contrast, “nanostructured,” “nanophase,” and “nanomaterial” refer to ceramics having a grain size of less than 100 nm in at least one direction. Mechanical properties, specifically, ductility and toughness, of these conventional biosubstitutes, however, are generally not comparable to natural bone. Consequently, use of these materials in orthopedic/dental applications has been limited. As one example, alumina has been used in the treatment of hand and elbow fractures, edentations, and in arthroplasty. There is therefore a need for biomaterials having ductility and toughness comparable to natural bone.

Implants composed of conventional ceramics have also experienced clinical failure. The cause of failure in the case of ceramic implants has been attributed to a lack of direct bonding with bone, that is, insufficient osseointegration. Osseointegration is necessary in order to stabilize orthopedic/dental prostheses in situ, to minimize motion-induced damage to surrounding tissues, and to increase overall implant efficacy. Insufficient bonding of juxtaposed bone to an orthopedic/dental implant can be caused by material surface properties that do not support new bone growth, as with implant materials composed of metal or conventional ceramics. The extent of osseointegration between bone and a newly implanted material is influenced by many factors including a number of host tissue responses. Physical and chemical properties of the biomaterial surface control the type and magnitude of cellular and molecular events at the tissue-implant interface.

Adhesion of bone-forming cells, or osteoblasts, to an implant is initially required for osseointegration. However, enhanced adhesion of osteoblasts to material surfaces does not necessarily result in enhancement of the long-term cell functions which lead to osseointegration of orthopedic/dental implants and, therefore, a successful implant. For example, Dee et al. (Biomaterials, 17 (2): pages 209-15 (1996)) immobilized RGDS (SEQ ID NO:15) (Arginine-Glycine-Aspartic Acid-Serine) peptides on glass. They observed enhanced osteoblast adhesion but not enhancement of subsequent functions, finding that mineralization on the peptide-modified glass was similar to that on unmodified glass. Osteoblast functions which occur subsequent to adhesion, and which are required for an effective implant, include proliferation, alkaline phosphatase synthesis, and deposition of extracellular matrix calcium. Enhancement of these long-term osteoblast functions on nanophase ceramics has not been reported. Therefore, there is a need for biomaterials having surface properties that enhance these and other long-term osteoblast functions. There is also a need for biomaterials with surface properties that would aid in the formation of new bone at the tissue/biomaterial interface and therefore, improve orthopedic/dental implant efficacy.

Synthetic peptides have been studied for their potential use in improving bone repair. To date, several biomolecules have been used, with the majority of them being proteins and peptide motifs. Encoded by specific amino acid sequences, these biomolecules target and bind specific cell surface receptors to trigger different intracellular signaling pathways. For example, with distinctive 3-dimensional conformation, peptide motifs such as RGDS (SEQ ID NO:15) have been shown to be mediators of cell adhesion and promote subsequent functions similar to the larger parental ECM proteins. In comparison with larger high-molecular-weight proteins, these relatively short peptides are resistant to denaturing (such as those caused by variations in pH, heat, and enzyme degradation). Also, these peptides can be synthesized with precise control of their chemical composition. Thus, there is the potential to develop small peptide-based therapeutics that function either as agonists to promote the interaction of cells and tissues with substrates, or as antagonists to control the nature of cell-cell and cell-ECM interactions.

Extensive studies over the past decade have been performed toward the incorporation of numerous cell adhesion promoting entities for various tissue engineering applications. While numerous cellular studies have provided some promising results for cell adherence, stability and pharmacological effects in vivo remain a major concern. There is little guidance for the selection of the many available strategies, many of which are labor intensive, require over several days to accomplish, and are expensive.

Therefore, there is a need for small peptide-based therapeutics that can be used in tissue engineering applications for bone augmentation.

The present invention is directed to overcoming these and other deficiencies in the art.

### SUMMARY OF THE INVENTION

In one aspect, the present invention relates a synthetic peptide. In one embodiment, the synthetic peptide of the present invention includes an amino acid sequence selected from the group consisting of the following:

(SEQ ID NO: 2)
KSRR,
(SEQ ID NO: 3)
KRSRGGGGY,
(SEQ ID NO: 4)
KSRRGGGGY,
(SEQ ID NO: 5)
KPSSAPTQLN,
(SEQ ID NO: 6)
AISVLYFDDS,
(SEQ ID NO: 7)
SNVILKKYRN,
(SEQ ID NO: 8)
KRSRSNVILKKYRN,
(SEQ ID NO: 9)
KRSRGGGGKPSSAPTQLN,
(SEQ ID NO: 10)
KRSRGGGGAISVLYFDDS,
(SEQ ID NO: 11)
KRSRGGGGSNVILKKYRN,
(SEQ ID NO: 12)
KRSRGGGGKPSSAPTQLNAISVLYFDDS,
(SEQ ID NO: 13)
KRSRGGGGAISVLYFDDSSNVILKKYRN,
and
(SEQ ID NO: 14)
KRSRGGGGKPSSAPTQLNAISVLYFDDSSNVILKKYRN,

or an amide, ester, or salt thereof. In a more particular embodiment, the synthetic peptide of the present invention can include terminal groups, including, for example, an N-terminal Ac group and/or a C-terminal CONH₂ group.

In another aspect, the present invention relates to a composition including: a synthetic peptide of the present invention; and a biocompatible material.

In another aspect, the present invention relates to a pharmaceutical composition for enhancing bone tissue repair or bone tissue regeneration, where the composition includes: a therapeutically effective amount of a synthetic peptide of the present invention; and a biocompatible carrier.

In another aspect, the present invention relates to an implantable prosthesis, where the implantable prosthesis includes: a prosthesis component coated with a composition of the present invention.

In another aspect, the present invention relates to an implantable orthopedic/dental device, where the device includes: an implantable substrate combined with a synthetic peptide of the present invention.

In another aspect, the present invention relates to a method for enhancing bone repair. This method involves contacting a site in need of repair tissue with a composition comprising a synthetic peptide of the present invention.

In another aspect, the present invention relates to a method of inducing osteogenesis. This method involves contacting bone cells with the synthetic peptide of the present invention, thereby inducing osteogenesis.

In another aspect, the present invention relates to a method for enhancing cell adhesion. This method involves bringing a cell into contact with a concentration of the synthetic peptide of the present invention sufficient to enhance cell adhesion.

In another aspect, the present invention relates to a method of constructing a bone replacement or bone-reconstructive material. This method involves preparing a biodegradable polymer matrix which incorporates a synthetic peptide of the present invention, and allowing osteoblasts to come into contact with the polymer matrix.

In another aspect, the present invention relates to a method for enhancing the stabilization of an implant. This method involves providing an implant with a coating of a synthetic peptide of the present invention.

In another aspect, the present invention relates to a bone replacement or bone-reconstructive material, which includes a polymer matrix and a synthetic peptide of the present invention.

In another aspect, the present invention relates to a method for promoting the adhesion of osteoblasts to a surface. This method involves: (a) providing a synthetic peptide of the present invention; (b) applying said peptide to a surface; and (c) bringing osteoblasts into contact with said surface, whereby the adhesion of said osteoblasts to said surface is enhanced.

In one aspect, the present invention disclosed herein produces peptide sequences mimicking natural protein bioactive portions that effectively promote the adhesion and density of corresponding tissue cells.

In comparison with larger high-molecular-weight proteins that traditionally used in clinical application for tissue response, the relatively short peptides discussed in this invention are resistant to denaturing (such as those caused by variations in pH, heat, and enzyme degradation). Also, these peptides can be synthesized with precise control of their chemical composition. Thus, there is the potential to develop small peptide-based therapeutics that function either as agonists to promote the interaction of cells and tissues with substrates, or as antagonists to control the nature of cell-cell and cell-ECM interactions.

These and other objects, features, and advantages of this invention will become apparent from the following detailed description of the various aspects of the invention taken in conjunction with the accompanying drawings.

### BRIEF DESCRIPTION OF THE DRAWINGS

For the purpose of illustrating aspects of the present invention, there are depicted in the drawings certain embodiments of the invention. However, the invention is not limited to the precise arrangements and instrumentalities of the embodiments depicted in the drawings. Further, as provided, like reference numerals contained in the drawings are meant to identify similar or identical elements.

FIG. 1 is a photograph illustrating a peptide solution in buffer and a test scaffold.

FIG. 2 is a graph of the results from an osteoblast proliferation study of various synthetic peptides of the present invention. The peptides (from left to right) correspond to the following: KRSR (SEQ ID NO:1), KSRR (SEQ ID NO:2), KRSRGGGGY (SEQ ID NO:3), KSRRGGGGY (SEQ ID NO:4), KPSSAPTQLN (SEQ ID NO:5), AISVLYFDDS (SEQ ID NO:6), SNVILKKYRN (SEQ ID NO:7), KRSRSNVILKKYRN (SEQ ID NO:8), KRSRGGGGKPSSAPTQLN (SEQ ID NO:9), KRSRGGGGAISVLYFDDS (SEQ ID NO:10), KRSRGGGGSNVILKKYRN (SEQ ID NO:11), KRSRGGGGKPSSAPTQLNAISVLYFDDS (SEQ ID NO:12), KRSRGGGGAISVLYFDDSSNVILKKYRN (SEQ ID NO:13), and KRSRGGGGKPSSAPTQLNAISVLYFDDSSNVILKKYRN (SEQ ID NO:14).

FIG. 3 is a graph of the results from an alkaline phosphatase activity study of various peptides of the present invention. The peptides (from left to right) correspond to the following: KRSR (SEQ ID NO:1), KSRR (SEQ ID NO:2), KRSRGGGGY (SEQ ID NO:3), KSRRGGGGY (SEQ ID NO:4), KPSSAPTQLN (SEQ ID NO:5), AISVLYFDDS (SEQ ID NO:6), SNVILKKYRN (SEQ ID NO:7), KRSRSNVILKKYRN (SEQ ID NO:8), KRSRGGGGKPSSAPTQLN (SEQ ID NO:9), KRSRGGGGAISVLYFDDS (SEQ ID NO:10), KRSRGGGGSNVILKKYRN (SEQ ID NO:11), KRSRGGGGKPSSAPTQLNAISVLYFDDS (SEQ ID NO:12), KRSRGGGGAISVLYFDDSSNVILKKYRN (SEQ ID NO:13), and KRSRGGGGKPSSAPTQLNAISVLYFDDSSNVILKKYRN (SEQ ID NO:14).

FIG. 4 is a graph of the results from a calcium deposition study of various peptides of the present invention. The peptides (from left to right) correspond to the following: KRSR (SEQ ID NO:1), KSRR (SEQ ID NO:2), KRSRGGGGY (SEQ ID NO:3), KSRRGGGGY (SEQ ID NO:4), KPSSAPTQLN (SEQ ID NO:5), AISVLYFDDS (SEQ ID NO:6), SNVILKKYRN (SEQ ID NO:7), KRSRSNVILKKYRN (SEQ ID NO:8), KRSRGGGGKPSSAPTQLN (SEQ ID NO:9), KRSRGGGGAISVLYFDDS (SEQ ID NO:10), KRSRGGGGSNVILKKYRN (SEQ ID NO:11), KRSRGGGGKPSSAPTQLNAISVLYFDDS (SEQ ID NO:12), KRSRGGGGAISVLYFDDSSNVILKKYRN (SEQ ID NO:13), and KRSRGGGGKPSSAPTQLNAISVLYFDDSSNVILKKYRN (SEQ ID NO:14).

FIG. 5 is a graph of the results from a total protein study of various peptides of the present invention. The peptides (from left to right) correspond to the following:

(SEQ ID NO: 1)
KRSR,
(SEQ ID NO: 2)
KSRR,
(SEQ ID NO: 3)
KRSRGGGGY,
(SEQ ID NO: 4)
KSRRGGGGY,
(SEQ ID NO: 5)
KPSSAPTQLN,
(SEQ ID NO: 6)
AISVLYFDDS,
(SEQ ID NO: 7)
SNVILKKYRN,
(SEQ ID NO: 8)
KRSRSNVILKKYRN,
(SEQ ID NO: 9)
KRSRGGGGKPSSAPTQLN,
(SEQ ID NO: 10)
KRSRGGGGAISVLYFDDS,
(SEQ ID NO: 11)
KRSRGGGGSNVILKKYRN,
(SEQ ID NO: 12)
KRSRGGGGKPSSAPTQLNAISVLYFDDS,
(SEQ ID NO: 13)
KRSRGGGGAISVLYFDDSSNVILKKYRN,
and
(SEQ ID NO: 14)
KRSRGGGGKPSSAPTQLNAISVLYFDDSSNVILKKYRN.

FIG. 6 is a graph showing histology data of various peptides of the present invention. Peptide 1=Ac-KRSR-NH₂ (SEQ ID NO:1). Peptide 2=Ac-KRSRSNVILKKYRN-NH₂ (SEQ ID NO:8).

FIG. 7 is a graph showing peptide conjugation and elution data for PLGA and HA based materials.

### DEFINITIONS

In describing and claiming the invention, the following terminology will be used in accordance with the definitions set forth below.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.”

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “peptide” encompasses a sequence of 3 or more amino acids wherein the amino acids are naturally occurring or synthetic (non-naturally occurring) amino acids. Peptide mimetics include peptides having one or more of the following modifications:
(i) peptides wherein one or more of the peptidyl —C(O)NR— linkages (bonds) have been replaced by a non-peptidyl linkage such as a —CH₂-carbamate linkage (—CH₂OC(O)NR—), a phosphonate linkage, a —CH₂-sulfonamide (—CH₂—S(O)₂NR—) linkage, a urea (—NHC(O)NH—) linkage, a —CH₂-secondary amine linkage, or with an alkylated peptidyl linkage (—C(O)NR—) wherein R is C₁-C₄ alkyl;
(ii) peptides wherein the N-terminus is derivatized to a —NRRi group, to a —NRC(O)R group, to a —NRC(O)OR group, to a —NRS(O)₂R group, to a —NHC(O)NHR group where R and R₁ are hydrogen or C₁-C₄ alkyl with the proviso that R and R₁ are not both hydrogen; or
(iii) peptides wherein the C terminus is derivatized to —C(O)R₂ where R₂ is selected from the group consisting of C₁-C₄ alkoxy, and —NR₃R₄ where R₃ and R₄ are independently selected from the group consisting of hydrogen and C₁-C₄ alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Val or V; Serine is Ser or S; Proline is Pro or P; Threonine is Thr or T; Alanine is Ala or A; Tyrosine is Tyr or Y; Histidine is H is or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

As used herein, the term “conservative amino acid substitution” is defined herein as exchanges within one of the following five groups: (i) Small aliphatic, nonpolar or slightly polar residues: Ala, Ser, Thr, Pro, Gly; (ii) Polar, negatively charged residues and their amides: Asp, Asn, Glu, Gln; (iii) Polar, positively charged residues: H is, Arg, Lys; (iv) Large, aliphatic, nonpolar residues: Met Leu, Ile, Val, Cys; and (v) Large, aromatic residues: Phe, Tyr, Trp.

As used herein, the term “solid support” relates to a solvent insoluble substrate that is capable of forming linkages (preferably covalent bonds) with soluble molecules. The support can be either biological in nature, such as, without limitation, a cell or bacteriophage particle, or synthetic, such as, without limitation, an acrylamide derivative, glass, plastic, agarose, cellulose, nylon, silica, or magnetized particles. The support can be in particulate form or a monolythic strip or sheet. The surface of such supports may be solid or porous and of any convenient shape.

As used herein, the term “purified” and like terms relate to the isolation of a molecule or compound in a form that is substantially free (at least 60% free, particularly 75% free, and most particularly 90% free) from other components normally associated with the molecule or compound in a native environment.

“Therapeutic agent,” “pharmaceutical agent” or “drug” refers to any therapeutic or prophylactic agent which may be used in the treatment (including the prevention, diagnosis, alleviation, or cure) of a malady, affliction, disease or injury in a patient.

As used herein, the term “treating” includes alleviating the symptoms associated with a specific disorder or condition and/or preventing or eliminating said symptoms.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “parenteral” includes administration subcutaneously, intravenously or intramuscularly.

The term “biocompatible,” as used herein, refers to a material that does not elicit a substantial detrimental response in the host.

As used herein the term “bioactive agent” refers to substances which are capable of exerting a biological effect in vitro and/or in vivo.

### DETAILED DESCRIPTION OF THE INVENTION

In one aspect, the present invention relates to the development of peptide sequences for use in treatments involving bone tissue repair and regeneration. For example, in one embodiment, by using peptides present in the tissue of interest as a bridging unit between cell receptors and a surface, different cellular pathways can be activated for subsequent biological responses.

According to one aspect, the present invention relates a synthetic peptide. In one embodiment, the synthetic peptide of the present invention includes an amino acid sequence selected from the group consisting of the following:

(SEQ ID NO: 2)
KSRR,
(SEQ ID NO: 3)
KRSRGGGGY,
(SEQ ID NO: 4)
KSRRGGGGY,
(SEQ ID NO: 5)
KPSSAPTQLN,
(SEQ ID NO: 6)
AISVLYFDDS,
(SEQ ID NO: 7)
SNVILKKYRN,
(SEQ ID NO: 8)
KRSRSNVILKKYRN,
(SEQ ID NO: 9)
KRSRGGGGKPSSAPTQLN,
(SEQ ID NO: 10)
KRSRGGGGAISVLYFDDS,
(SEQ ID NO: 11)
KRSRGGGGSNVILKKYRN,
(SEQ ID NO: 12)
KRSRGGGGKPSSAPTQLNAISVLYFDDS,
(SEQ ID NO: 13)
KRSRGGGGAISVLYFDDSSNVILKKYRN,
and
(SEQ ID NO: 14)
KRSRGGGGKPSSAPTQLNAISVLYFDDSSNVILKKYRN,

or an amide, ester, or salt thereof.

In another embodiment, the synthetic peptide of the present invention can include terminal groups, including, for example, an N-terminal Ac group and/or a C-terminal CONH₂ group.

In one embodiment, the synthetic peptide of the present invention can further include a bioactive agent linked to the synthetic peptide. In one embodiment, the bioactive agent is covalently bound to the synthetic peptide. Suitable bioactive agents can include, for example, chemotherapeutics and nucleic acid sequences. Suitable bioactive agents can also include, without limitation, agents such as hydroxyapatite (HA) and the like.

Thus, in accordance with one embodiment, the synthetic peptides of the present invention are complexed or linked to one or more bioactive agents. The bioactive agents can be linked to the bone targeting peptides through hydrogen, ionic, or covalent bonding. In one embodiment the bioactive agent is covalently linked to a peptide of the present invention. Also in accordance with this invention is the use of indirect means for associating the bioactive agents with the peptides including by connection through intermediary linkers, spacer arms, bridging molecules, or liposome entrapment. In one embodiment, the peptide/bioactive agent complex can be used to deliver therapeutic pharmaceuticals to bone or cartilage tissues, wherein the bioactive agents are encapsulated within the liposome. Bioactive agents suitable for use with the present invention can include, without limitation, antibodies, growth factors, toxins (such as aflatoxin, digoxin, xanthotoxin, rubratoxin), antibacterial agents (such as cephalosporins, penicillin, erythromycin, ciprofloxacin, cinoxacin, and norfloxacin), cancer drugs (including chemotherapeutic agents), and nucleic acids. In one embodiment the bone targeting protein is linked to a chemotherapeutic agent or other cancer drug and the complex is used to treat a patient suffering from cancer, especially bone cancer or cancer that has metastasized to bone or cartilagenous tissues.

The present invention further relates to bioactive fragments and derivatives of the peptides of SEQ ID NOS:2-14. Derivatives of SEQ ID NOS:2-14 can include amino acid sequences that differ from those sequences either by one or more conservative amino acid substitutions, or by one amino acid deletion, addition or substitution. In one embodiment, the peptides comprise a sequence identical to SEQ ID NO:2-SEQ ID NO:14, or differ from SEQ ID NO:2-SEQ ID NO:14 by 1-2 conservative amino acids.

In one embodiment, the peptides of the present invention can be prepared from natural proteins, produced recombinantly, or more particularly they are chemically synthesized using techniques well known to those of ordinary skill in the art. The present invention is also directed to antibodies that specifically bind to a peptide selected from the group consisting of SEQ ID NOs:2-14.

In another aspect, the present invention relates to a composition including: a synthetic peptide of the present invention; and a biocompatible material. A suitable biocompatible material can include, without limitation, a pharmaceutically acceptable carrier, a polymer matrix, a tissue scaffold, a delivery vehicle, and the like. As one example, a suitable delivery vehicle can be a biodegradable polymer. Suitable biocompatible materials can also include, without limitation, an allograft, a demineralized bone matrix, collagen, a xenograft, and the like.

In another aspect, the present invention relates to a pharmaceutical composition for enhancing bone tissue repair or bone tissue regeneration, where the composition includes: a therapeutically effective amount of a synthetic peptide of the present invention; and a biocompatible carrier. In one embodiment, the carrier can be a single dose carrier. In another embodiment, the carrier can be a collagen matrix.

As noted herein, aspects of the present invention encompass pharmaceutical and therapeutic compositions that include the synthetic peptides of the present invention. In accordance with one embodiment, the present invention is directed to a composition comprising a purified peptide comprising a sequence identical to SEQ ID NO:2-SEQ ID NO:14, or differing from SEQ ID NO:2-SEQ ID NO:14 by 1-2 conservative amino acids, and a biocompatible material. In one embodiment, the biocompatible material constitutes a pharmaceutically acceptable carrier. Alternatively, the biocompatible material may comprise a solid carrier or polymer matrix, wherein a peptide of the present invention is entrapped within the carrier or matrix or otherwise bound to the surface of the carrier or matrix. In one embodiment, the composition comprises a peptide of the present invention and a bioresorbable/biodegradeable polymer matrix, wherein the polymer matrix provides timed release of the bioactive peptides.

Many matrix systems have been developed to contain and then steadily release bioactive peptides as the matrix degrades. For example, organic polymers such as polylactides, polyglycolides, polyanhydrides, and polyorthoesters, which readily hydrolyze in the body into inert monomers, have been used as matrixes (see U.S. Pat. Nos. 4,563,489; 5,629,009; and 4,526,909, which are incorporated by reference in their entirety). The efficiency of peptide release from polymer matrixes depends on the matrixes resorbtion rate, density, and pore size. Monomer type and their relative ratios in the matrix influence these characteristics. Polylactic and polyglycolic acid copolymers, protein sequestering agents, and osteoinductive factors provide the necessary qualities for a bioactive peptide delivery system (see U.S. Pat. No. 5,597,897, which is incorporated by reference in its entirety). Alginate, poly(ethylene glycol), methyl methacrylate, polyoxyethylene oxide, carboxyvinyl polymer, and poly (vinyl alcohol) are additional polymer examples that can be used in accordance with the present invention.

Non-synthetic matrix proteins like collagen, glycosaminoglycans, and hyaluronic acid, which are enzymatically digested in the body, have also been used to deliver bioactive proteins to bone areas (see U.S. Pat. Nos. 4,394,320; 4,472,840; 5,366,509; 5,606,019; 5,645,591; and 5,683,459, which are incorporated by reference in their entirety) and are suitable for use with the present invention. The bone targeting peptide compositions can be further combined with a demineralized bone material, growth factor, nutrient factor, pharmaceutical, calcium-containing compound, anti-inflammatory agent, antimicrobial agent, or any other substance capable of expediting or facilitating bone growth. Examples of osteoinductive factor suitable for use with the compositions of the present invention include demineralized bone particles, a Bone Morphogenetic Protein, an osteoinductive extract of demineralized bone matrix, or a combination thereof.

Examples of growth factors suitable for use with the composition of the present invention include Transforming Growth Factor-Beta (TGF-β), Transforming Growth Factor-Alpha (TGF-α), Epidermal Growth Factor (EGF), Insulin Like Growth Factor-I or II, Interleukin-1 (IL-1), Interferon, Tumor Necrosis Factor, Fibroblast Growth Factor (FGF), Platelet Derived Growth Factor (PDGF), and Nerve Growth Factor (NGF).

The compositions of the present invention can also be combined with inorganic fillers or particles. For example, for use in implantable grafts the inorganic fillers or particles can be selected from hydroxyapatite, tri-calcium phosphate, ceramic glass, amorphous calcium phosphate, porous ceramic particles or powders, mesh titanium or titanium alloy, or particulate titanium or titanium alloy.

In another aspect, the present invention relates to an implantable prosthesis, where the implantable prosthesis includes: a prosthesis component coated with a composition of the present invention.

In another aspect, the present invention relates to an implantable orthopedic/dental device, where the device includes: an implantable substrate combined with a synthetic peptide of the present invention. In one embodiment, the substrate is combined with the synthetic peptide by covalent bonds. Suitable substrates can include, for example, ceramics, metals, polymers, and composites.

More particularly, one aspect of the present invention relates to osteogenic devices, and more specifically to synthetic implants which induce osteogenesis in vivo in mammals, including humans. More particularly, this embodiment of the invention relates to biocompatible, bioresorbable, synthetic compositions comprising the synthetic peptides disclosed herein. These compositions are anticipated to have osteogenic properties and/or are trophic for osteogenic cell images. The implants can be prepared using previously described implant materials such as hydroxlapatite, autogenous bone grafts, allogenic bone matrix, demineralized bone powder, collagenous matrix. The synthetic peptides of the present invention can be combined with known graft materials that are fully formable at temperatures above about 38° C., but become a solid at temperatures below about 38° C. Such compositions such as Opteform 100HT (University of Florida Tissue Bank) comprise a thermoplastic human derived inert carrier allowing the material stays rigid once it reaches body temperature. In another embodiment, the synthetic peptides are combined with known materials to provide a composition for coating implantable prosthetic devices, and to increase the cellular ingrowth into such devices.

In another aspect, the present invention relates to a method for enhancing bone repair. This method involves contacting a site in need of repair tissue with a composition comprising a synthetic peptide of the present invention. In one embodiment, the composition is in an injectable form, and the step of contacting the site involves administering the composition locally by injection. In another embodiment, the step of contacting the site involves surgically implanting the composition.

In another aspect, the present invention relates to a method of inducing osteogenesis. This method involves contacting bone cells with the synthetic peptide of the present invention, thereby inducing osteogenesis.

In another aspect, the present invention relates to a method for enhancing cell adhesion. This method involves bringing a cell into contact with a concentration of the synthetic peptide of the present invention sufficient to enhance cell adhesion.

In another aspect, the present invention relates to a method of constructing a bone replacement or bone-reconstructive material. This method involves preparing a biodegradable polymer matrix which incorporates a synthetic peptide of the present invention, and allowing osteoblasts to come into contact with the polymer matrix.

In another aspect, the present invention relates to a method for enhancing the stabilization of an implant. This method involves providing an implant with a coating of a synthetic peptide of the present invention.

In another aspect, the present invention relates to a bone replacement or bone-reconstructive material, which includes a polymer matrix and a synthetic peptide of the present invention. In one embodiment, the polymer is biodegradable. In another embodiment, the polymer is insert. In other embodiments, the bone replacement or bone-reconstructive material of the present invention can include, for example, allografts, demineralized bone matrices, collagen, and xenografts.

In another aspect, the present invention relates to a method for promoting the adhesion of osteoblasts to a surface. This method involves: (a) providing a synthetic peptide of the present invention; (b) applying said peptide to a surface; and (c) bringing osteoblasts into contact with said surface, whereby the adhesion of said osteoblasts to said surface is enhanced.

### EXAMPLES

The following examples are intended to illustrate particular embodiments of the present invention, but are by no means intended to limit the scope of the present invention.

### Example 1

### Development and Testing of Peptides for Bone Augmentation Applications

In the context of early testing of manufacturing, we have produced several novel peptide sequences that have better in vitro and in vivo responses over peptides currently available. Direct application of the peptide sequences (shown in Table 1) can be used in orthopedic, spine and dental use to promote implants and hard tissue interaction.

For example, in one series of studies we designed 14 peptide sequences for potential use in bone augmentation applications, as shown in Table 1 below.

TABLE 1

| SEQ | |
|---|---|
| ID | |
| NO: | PEPTIDE SEQUENCE |
| 1 | KRSR |
| 2 | KSRR |
| 3 | KRSRG GGGY |
| 4 | KSRRG GGGY |
| 5 | KPSSA PTQLN |
| 6 | AISVL YFDDS |
| 7 | SNVIL KKYRN |
| 8 | KRSRS NVILK KYRN |
| 9 | KRSRG GGGKP SSAPT QLN |
| 10 | KRSRG GGGAI SVLYF DDS |
| 11 | KRSRG GGGSN VILKK YRN |
| 12 | KRSRG GGGKP SSAPT QLNAI SVLYF DDS |
| 13 | KRSRG GGGAI SVLYF DDSSN VILKK YRN |
| 14 | KRSRG GGGKP SSAPT QLNAI SVLYF DDSSN VILKK |
| | YRN |

The peptide sequences of Table 1 contain components of KRSR-based sequences and BMP-7 fragments, as well as the combination of both. Glycine was used as spacers in some of the sequences. Since Glycine is a simple amino acid, it is anticipated that such modifications will not disturb the biological function of the designed peptide.

In vitro data with osteoblast results provided increased bone cell response and functions on a petri dish. This information was used to perform animal experiments with a mini-pig model. For this purpose, we used these peptides with Nanovis Bone Void Filler Materials (PLGA, Collagen, and NHA-based bone void filler materials) to determine the animal responses. Interestingly, we observed the similar trend of responses with some of these peptides in pig models. Thus, these peptide sequences were targeted for further clinical studies.

### Example 2

### Peptide Conjugation/Elution Strategy

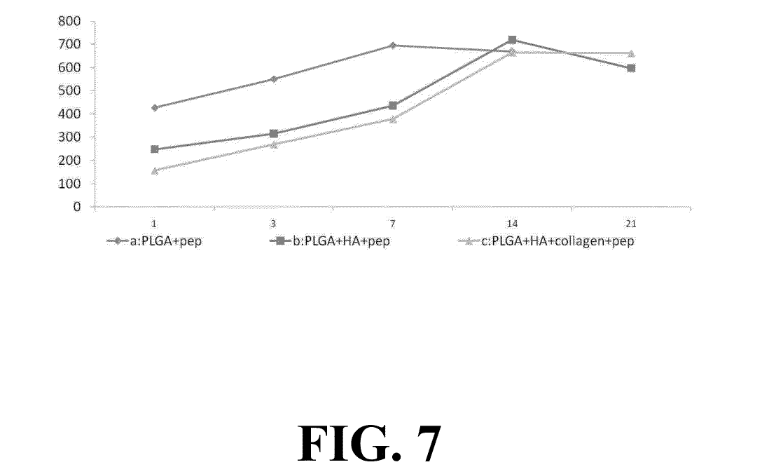

As shown in FIG. 7, peptide conjugation and elution strategies were identified for PLGA and HA based materials. It is possible that one can achieve a peptide elution profile ranging from hours (quick) to days (Medium to longer).

### Example 3

### Peptide Concentration Selection

Based on our preliminary animal experiment with PIG models, and our understanding, we will use a peptide concentration of in the range of 10⁻¹² M to 10⁻⁶ M for cellular response. Weight of each peptide is labeled by manufacturer on every vial. Based on that weight, we should able to prepare the desired concentration.

### Example 4

### Test Elution Methodology

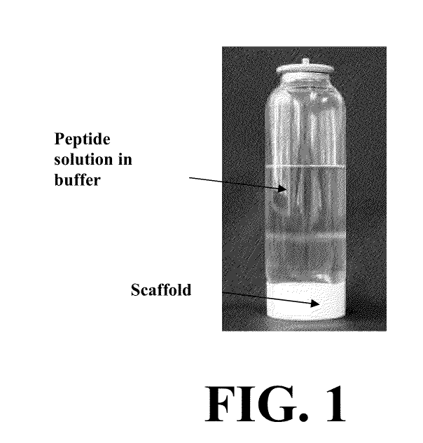

We have to use chromophore conjugated peptides for this particular test elution study for scaffolds. FIG. 1 shows a peptide solution in buffer and a scaffold. Alternatively, we can use Tyr containing peptide fragments. In vitro peptide release profiles from peptide bounded scaffolds will be determined as follows. Scaffolds will be suspended in 1 ml phosphate buffered saline (PBS, 10 mM, pH=7.4). The scaffold suspension will be incubated at 37° C. with an orbital shaker. At designated time points, samples will be centrifuged to collect supernatant replaced with equal amounts of fresh medium. UV absorbance will be measured using spectrophotometer using lcm path length cuvette. PBS will be referenced prior to measurement. The data will be converted to calculate the quantity of the released peptide. Blank scaffold (without any peptide) will be used as control.

If we use full protein, we can use BCA protein assay test.

### Example 5

### In Vitro and In Vivo Testing of Peptides

### Cell Adhesion Testing

Human fetal osteoblasts (hFOB, CRL-11372, ATCC) of population numbers 7-11 were cultured in Dulbecco's Modified Eagle's Medium (DMEM, GIBCO) supplemented with 10% fetal bovine serum (FBS, Hyclone) and 1% penicillin/streptomycin (P/S, Hyclone) under standard cell culture conditions (that is, a humidified, 5% CO₂/95% air environment at 37° C.). Osteoblasts were seeded onto the petri-dish at a density of 3500 cells/cm² and were cultured under standard cell culture conditions for 4 h with peptide concentration discussed. After the prescribed time period, non-adherent cells were removed by sequential phosphate buffered saline (PBS) washings. The remaining cells were fixed using 10% normal buffered formaldehyde (Fisher Scientific) for 10 min and 0.1% Triton X-100 (Sigma-Aldrich) for 5 min. Adherent cells were counted in five random fields per substrate under a Zeiss Axiovert 200M fluorescence microscope. Experiments were run in triplicate and repeated three separate times for each substrate.

### Osteoblast Proliferation Testing

For osteoblast proliferation studies, similar to the above procedures, osteoblasts were seeded on petri dish at 2500 cells/cm² with peptides and cultured for 1, 3 days. After the prescribed time periods, adherent cells were fixed, stained and counted under the fluorescence microscope as described above. All cellular experiments were run in triplicate and repeated at least three times for each substrate.

### Results

### Cell Adhesion and Osteoblast Proliferation Testing

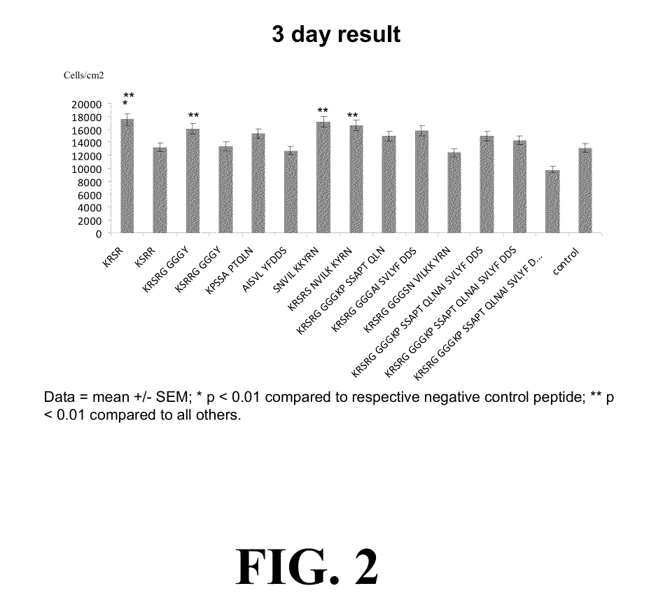

The KRSR (SEQ ID NO:1) sequence exhibited bioactivity that was a function of structural aspects of the peptide, was cell specific, and proved to be crucial for maximal osteoblast adhesion to substrates. After day 3 (see FIG. 2), osteoblast density on peptide coated surfaces showed interesting results. In particular, KRSR (SEQ ID NO:1), SNVILKKYRN (SEQ ID NO:7), and KRSRSNVILKKYRN (SEQ ID NO:8) increased osteoblast attachment compared to other peptides studied. Initially, we thought Glycine spacers would improve the peptide function for KRSR (SEQ ID NO:1) and BMP-7 fragments. However, in contrast, it appears that the glycine units allow the KRSR (SEQ ID NO:1) fragments to fold back into the BMP-7 fragments and interfere with its functionality. This is very useful information to understand the importance of introducing spacers in such applications.

### Example 6

### Cell Function Tests

Osteoblast Function

Total intracellular protein content of osteoblasts is extremely important since it is the indication of healthy growth and normal cell response. Thus, the amount of protein produced by cells were measured up to 3 weeks of culture after providing complete media. In order to release the intracellular protein, the adhered cells on the substrates were lysed in DI water using a standard four cycle freeze-thaw method. The resulting lysate solution was then used for analysis. The total protein content were determined by a BCA (bicinchoninic acid) assay kit (Pierce) and the absorbance of the solution were measured using a spectrophotometer at a wavelength of 570 nm. The absorbance was then converted to protein content using an albumin standard curve to determine the amount of intracellular protein.

Alkaline Phosphatase Activity

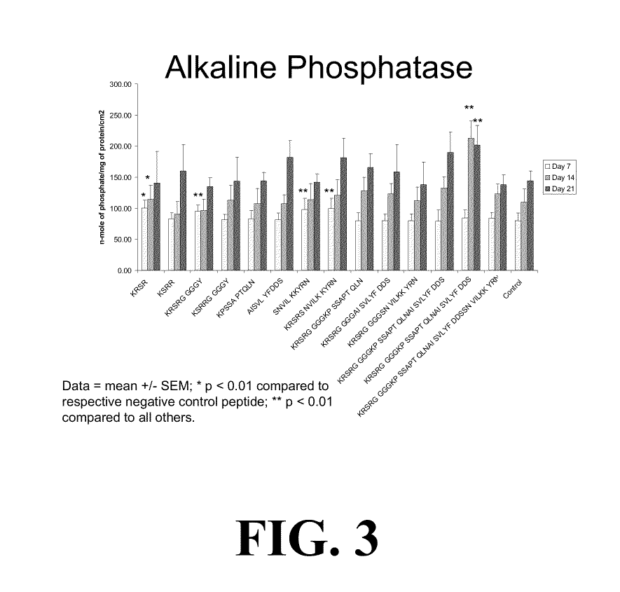

Alkaline phosphatase activity (ALP) is an important parameter to access the normal functionality of osteoblasts on a surface; hence, the activity was measured up to 3 weeks after providing complete media (see FIG. 3). The resulting lysate solution obtained by a four cycle freeze-thaw method was used to measure the ALP activity using a colorimetric assay (Teco). The absorbance of the solution was measured using a spectrophotometer at a wavelength of 590 nm. The absorbance was converted to concentration using ALP standard and all the data were normalized with total protein content to account for changes in number of cells present on surface.

Calcium Content

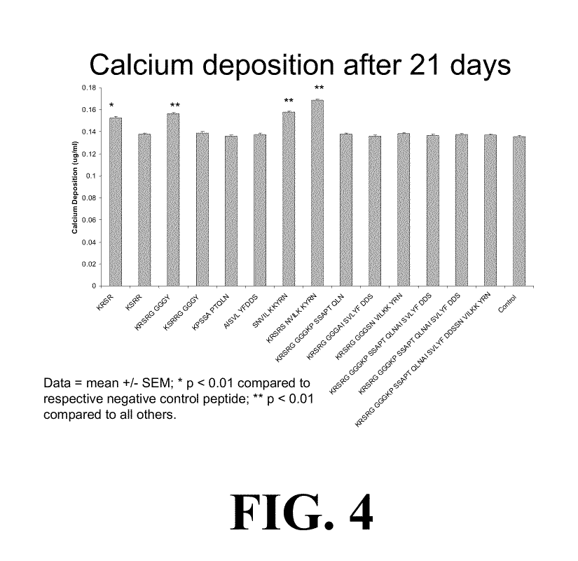

The calcium content was measured up to 3 weeks in culture using a colorimetric assay (Teco) (see FIG. 4). After all the lysate was aspirated, the surfaces were soaked overnight in 6 N HCl solution to dissolve the deposited calcium. The calcium solution was then reacted with assay reagents and the absorbance of the solution was measured photometrically at 570 nm. The absorbance was then converted to concentration using calcium standards and all the data were normalized with total protein content to account for changes in the number of cells present on surface.

Total Proteins

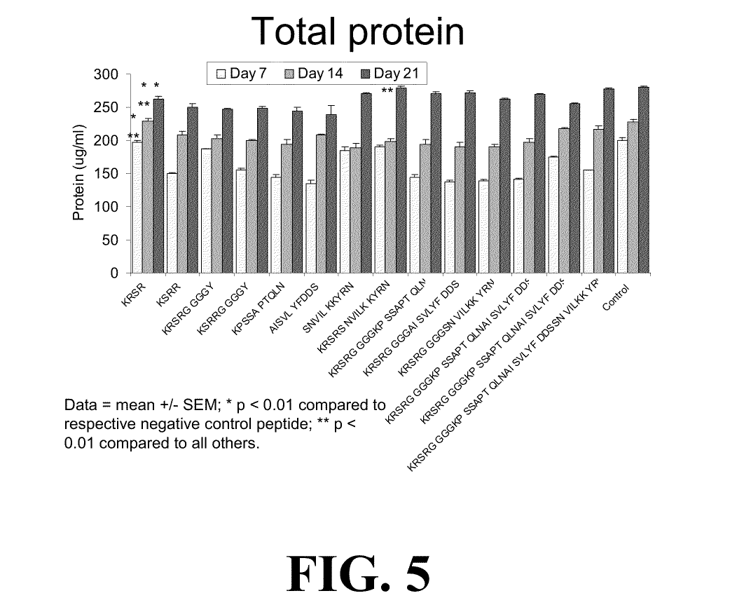

Total proteins synthesized by osteoblasts were greater when cultured on uncoated substrates compared to peptide coatings after 7 and 14 days (see FIG. 5). In particular, KRSRSNVILKKYRN (SEQ ID NO:8) showed the highest value compared to other peptides and the control. The greatness of KRSRSNVILKKYRN (SEQ ID NO:8) was further supported by the calcium deposition studies. After 21 days, KRSRSNVILKKYRN (SEQ ID NO:8) showed highest value compared to other materials studied (see FIG. 5). Further alkaline phosphatase data revealed the superiority of the KRSRSNVILKKYRN (SEQ ID NO:8) compared to other peptides. Overall, KRSR (SEQ ID NO:1) and KRSRSNVILKKYRN (SEQ ID NO:8) showed increased osteoblast function. Further studies can be conducted to further evaluate their use in bone applications.

### Example 7

### Animal Study Design

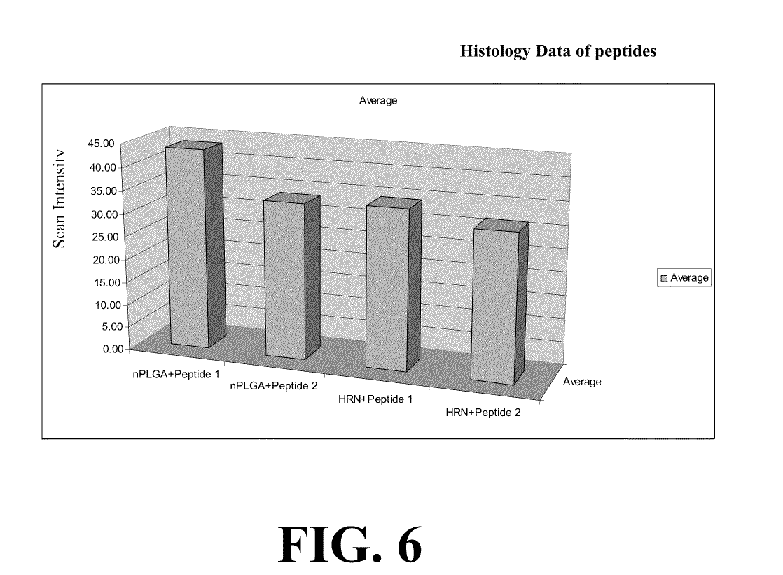

Animal study data revealed that both peptides KRSR (SEQ ID NO:1) and KRSRSNVILKKYRN (SEQ ID NO:8) showed new bone growth with the scaffolds studied (nPLGA and HRN based) (see FIG. 6).

A critical sized bone defect model was used in the proximal tibia and distal femur of 12 Sinclair minipigs (Sinclair Research, MO). Time points to be evaluated are 4 weeks and 8 weeks. Four surgical defects (2 tibia and 2 femur) were created on day 0 in all pigs in one limb and on day 28 in all pigs in the opposite limb. Treatments were applied according to predetermined treatment allocations. MRI evaluations of defects were performed at 4 weeks (all limbs) and 8 weeks (initial defects created on day 0). Following euthanasia, defects were evaluated using microCT and histology to measure bone volume fraction within the defects and with mechanical testing to evaluate compressive stiffness and integration with surrounding bone.

### Example 8

### Animal Study Procedures

### Critical Sized Bone Defect Surgery

Minipigs were given pre-operative analgesic and antimicrobial drugs immediately prior to anesthesia. Pigs were anesthetized in the Clinical Discovery Laboratory (CDL) using standard swine protocols. One stifle region was aseptically prepared and draped routinely for surgery of the proximal tibia and distal femur. Defects were created in epiphyseal/metaphyseal cancellous bone using an 8 mm drill to a depth of 20 mm. The procedure was repeated in the opposite hind limb at 4 weeks. (Confirmation of the critical defect size in minipigs is to be determined.)

### Post-Operative Monitoring and Euthanasia

Minipigs were housed in the animal holding facility room 1094 and monitored post-operatively. Euthanasia was performed using an overdose of barbiturate following induction of anesthesia using a telazol/xylazine mixture. Tissue/implant harvesting were performed immediately following MRI/radiographic evaluations.

### Tissue Processing

Bone tissue segments were harvested for mechanical testing, microCT and histology (see FIG. 6). With regard to FIG. 6, Peptide 1=Ac-KRSR-NH₂ (SEQ ID NO:1) and Peptide 2=Ac-KRSRSNVILKKYRN-NH₂ (SEQ ID NO:8).

### Example 9

### Further Studies

Further studies can be conducted on the peptides of the present invention in preclinical trials, including complete in vitro cell cytocompatability and cell function tests and subsequently in vivo animal models mainly for repairing orthopedic, cartilage, dental, spine, and soft tissue repair applications.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A synthetic peptide comprising an amino acid sequence selected from the group consisting of:
(SEQ ID NO: 2)
KSRR,
(SEQ ID NO: 3)
KRSRGGGGY,
(SEQ ID NO: 4)
KSRRGGGGY,
(SEQ ID NO: 5)
KPSSAPTQLN,
(SEQ ID NO: 6)
AISVLYFDDS,
(SEQ ID NO: 7)
SNVILKKYRN,
(SEQ ID NO: 8)
KRSRSNVILKKYRN,
(SEQ ID NO: 9)
KRSRGGGGKPSSAPTQLN,
(SEQ ID NO: 10)
KRSRGGGGAISVLYFDDS,
(SEQ ID NO: 11)
KRSRGGGGSNVILKKYRN,
(SEQ ID NO: 12)
KRSRGGGGKPSSAPTQLNAISVLYFDDS,
(SEQ ID NO: 13)
KRSRGGGGAISVLYFDDSSNVILKKYRN,
and
(SEQ ID NO: 14)
KRSRGGGGKPSSAPTQLNAISVLYFDDSSNVILKKYRN,
or an amide, ester, or salt thereof.

2. The synthetic peptide according to claim 1 further comprising:
a bioactive agent linked to said synthetic peptide.

3. The synthetic peptide according to claim 2, wherein the bioactive agent is covalently bound to said synthetic peptide.

4. The synthetic peptide according to claim 2, wherein the bioactive agent is selected from the group consisting of chemotherapeutics and nucleic acid sequences.

5. The synthetic peptide according to claim 2, wherein the bioactive agent comprises hydroxyapatite (HA) or the like.

6. The synthetic peptide according to claim 1 further comprising:
a terminal group attached to the N terminus and/or C terminus of the synthetic peptide, said terminal group being selected from the group consisting of an N-terminal Ac group and a C-terminal CONH₂ group.

7. A composition comprising:
a synthetic peptide according to claim 1; and
a biocompatible material.

8. The composition according to claim 7, wherein the biocompatible material comprises a pharmaceutically acceptable carrier.

9. The composition according to claim 7, wherein the biocompatible material is a polymer matrix.

10. The composition according to claim 7, wherein the biocompatible material is a tissue scaffold.

11. The composition according to claim 7, wherein the biocompatible material comprises a delivery vehicle.

12. The composition according to claim 11, wherein the delivery vehicle is a biodegradable polymer.

13. The composition according to claim 7, wherein the biocompatible material is selected from the group consisting of an allograft, a demineralized bone matrix, collagen, and a xenograft.

14. A pharmaceutical composition for enhancing bone tissue repair or bone tissue regeneration, said composition comprising:
a therapeutically effective amount of a synthetic peptide according to claim 1; and
a biocompatible carrier.

15. The pharmaceutical composition according to claim 14, wherein the carrier is a single dose carrier.

16. The pharmaceutical composition according to claim 14, wherein the carrier is a collagen matrix.

17. An implantable prosthesis comprising:
a prosthesis component coated with a composition according to claim 7.

18. An implantable orthopedic/dental device comprising:
an implantable substrate combined with the synthetic peptide according to claim 1.

19. The device according to claim 18, wherein the substrate is combined with the synthetic peptide by covalent bonds.

20. The device according to claim 18, wherein the substrate is selected from the group consisting of ceramics, metals, polymers, and composites.

21. A method for enhancing bone repair, said method comprising:
contacting a site in need of repair tissue with a composition comprising the synthetic peptide according to claim 1.

22. The method according to claim 21, wherein the composition is in an injectable form, and wherein the step of contacting the site comprises administering the composition locally by injection.

23. The method according to claim 21, wherein the step of contacting the site comprises surgically implanting the composition.

24. A method of inducing osteogenesis, said method comprising:
contacting bone cells with the synthetic peptide according to claim 1, thereby inducing osteogenesis.

25. A method for enhancing cell adhesion, said method comprising:
bringing a cell into contact with a concentration of the synthetic peptide according to claim 1 sufficient to enhance cell adhesion.

26. A method of constructing a bone replacement or bone-reconstructive material, said method comprising:
preparing a biodegradable polymer matrix which incorporates the synthetic peptide according to claim 1, and
allowing osteoblasts to come into contact with the polymer matrix.

27. A method for enhancing the stabilization of an implant, said method comprising:
providing an implant with a coating of a synthetic peptide according to claim 1.

28. A bone replacement or bone-reconstructive material comprising:
a polymer matrix and a synthetic peptide according to claim 1.

29. The bone replacement or bone-reconstructive material according to claim 28, wherein the polymer is biodegradable.

30. The bone replacement or bone-reconstructive material according to claim 28, wherein the polymer is inert.

31. A method for promoting the adhesion of osteoblasts to a surface, said method comprising:
(a) providing a synthetic peptide according to claim 1;
(b) applying said peptide to a surface; and
(c) bringing osteoblasts into contact with said surface, whereby the adhesion of said osteoblasts to said surface is enhanced.

32. A method of preparing an implantable biomaterial composition, said method comprising:
(a) providing a synthetic peptide according to claim 1;
(b) combining the synthetic peptide with hydroxyapatite (HA) while wet under conditions to attach the synthetic peptide to nano HA particles;
(c) mixing the combined synthetic peptide-HA with other compositions such as a polymer to yield an implantable biomaterial composition.

33. A method of preparing an implantable biomaterial composition, said method comprising:
(a) providing a composition according to claim 7;
(b) configuring the composition into a paste to yield an implantable biomaterial composition.

34. The method according to claim 33, wherein said implantable biomaterial composition is suitable for use as a material selected from the group consisting of a material for use inside of a fusion cage, a material for use as a bone void filler, a material for use in treating a non-union fracture, and a material for injecting into zones of pseudoarthrosis.

35. An implantable biomaterial composition prepared by the method according to claim 33.
